(19) United States
(12) Patent Application Publication
(10) Pub. No.: US 2004/0242974 A1
(43) Pub. Date: Dec. 2, 2004

(54) Treatment of refractory depression with an opiate antagonist and an antidepressant

(76) Inventor: Hillel Glover, New York, NY (US)

Correspondence Address: DICKSTEIN SHAPIRO MORIN & OSHINSKY LLP, 2101 L STREET NW, WASHINGTON, DC, 20037-1526, US

(21) Appl. No.: 10/878,285

(22) Filed: Jun. 29, 2004

Related U.S. Application Data
(63) Continuation-in-part of application No. 09/925,190, filed on Aug. 9, 2001, now abandoned.

(51) Int. Cl. A61B 5/00; G06F 19/00; G01N 33/48; G01N 33/50

(52) U.S. Cl. 600/300; 702/19

(57) ABSTRACT

An antidepressant or a pharmaceutically acceptable salt thereof, and an opiate antagonist or a pharmaceutically acceptable salt thereof, are used to treat refractory depression characterized by dissociation.

### CROSS-REFERENCE TO OTHER APPLICATION

This application is a continuation-in-part of application Ser. No. 09/925,190, filed Aug. 9, 2001, the entirety of which in incorporated herein by reference.

### FIELD OF THE INVENTION

The present invention relates to the use of opiate antagonist and an antidepressant to treat psychopathologic conditions, more particularly, the use of an opiate antagonist and an antidepressant to treat refractory depression characterized by dissociation and other psychopathologic conditions.

### BACKGROUND OF THE INVENTION

The use of opiate antagonists to treat psychological conditions is known and has been used by mental health practitioners, as is well known to those skilled in the mental health art. The combined use of such opiate antagonist with antidepressants has also been demonstrated to treat depression. The combination, however, has not been heretofore proposed or used to solve problems of treating refractory depression characterized by dissociation, as we presently understand the prior art.

This invention relates to a method of treating refractory depression. It relates particularly to a method of treating refractory depression characterized by dissociation by administering to a patient at least one opiate antagonist, as well as an antidepressant. The invention further relates to treating refractory depression characterized by dissociation by administering to a patient in need thereof at least one opiate antagonist, evaluating the patient for a response to the opiate antagonist, reassessing the patient for depression, and administrating at least one antidepressant to the patient.

As used herein, treatment refractory depression and treatment-resistant depression are synonymous. Refractory depression means depressions that respond insufficiently to treatment with the standard antidepressants, such as tricyclic antidepressants (TCAs), monoamine oxidase inhibitors (MAOIs), and serotonin selective reuptake inhibitors (SSRIs), given long enough and in adequate dosage.

Opiate antagonists are useful in therapy for depression. U.S. Pat. Nos. 5,512,593, 5,817,665, and 5,856,332 cover such uses. They also disclose the use of opioid antagonists in combination with lithium and/or a tricyclic antidepressant and/or an a-typical antidepressant with and without concomitant administration of an anti-anxiety agent to treat emotional or mental illness or emotional or mental illness concomitant with an illness causing seizure. Also, the use of naltrexone is disclosed in combination with lithium and/or one or more serotonin (5-HT) uptake inhibitor and/or norepinephrine (N.E.) uptake inhibitor drug compounds in treating patients whose depression and/or associated mental illnesses or conditions were refractory to drug treatment using one or more known antidepressant agents or agents for manic and manic depressive disorders such as lithium, and tricyclic and a-typical antidepressants. The patents describe various uses of opiate antagonists with antidepressants to treat depression. These patents are hereby incorporated by reference.

A variety of interventions are also known in the art that deal with patients identified or diagnosed with refractory depression. These include adding additional antidepressants from other classes, steroid suppression therapy, augmentation with atypical antipsychotics and psychotherapy, augmentation with lithium and thyroid T3, partial sleep deprivation, and electroconvulsive therapy. Lithium augmentation seems to be the treatment strategy that has been investigated most frequently in placebo controlled double-blind studies of patients identified to have refractory depression. Despite this fact, there do not seem to be any specific prognostic indictors of long-term outcome to lithium augmentation. Electroconvulsive therapy (ECT) is one of the most effective biological treatments for major depression. Yet, no significant clinical predictor of ECT outcome has been found. Medication resistance was also found not to be related to ECT response.

Upwards of 30% to 45% of depressed patients who are treated with antidepressants show only partial or no response. Even among patients who are considered responders, there may be residual symptoms of depression. The presence of residual symptoms has been associated with a poorer prognosis and higher risk of relapse. Refractory depression is not a clinical entity, it is identified only on the basis of a poor clinical outcome. The clinician should examine potential factors that may contribute to an apparent non-response including the adequacy of the trial, patient compliance with medication, differential diagnosis, and treatable comorbid conditions. After addressing these variables, a patient who does not demonstrate a remission may be considered treatment resistant, relative or absolute.

Many people who are diagnosed with depression also dissociate. The essential features of dissociative disorders include the disruption in the usual integrative functions of consciousness, memory, identity, or perception of the environment. The disturbances may be sudden or gradual, transient or chronic. People who feel emotionally numb, dead, shut-down, hollow, empty, or who report that they cannot experience feelings have lost the ability to access normal human feelings as part of their conscious waking existence. Intimately associated with this disruption in their conscious experience of human feelings is the disruption in their experience of who they are; their sense of identity. People diagnosed with one of the dissociative disorders typically experience having no feelings. For example, feeling dead has been noted in people with symptoms of depersonalization. People diagnosed with dissociative identity disorder have been reported to have symptoms of numbness. The diagnostic criteria for 308.3 Acute Stress Disorder in the DSM IV (pp. 431-432), lists a subjective sense of numbing, detachment, or absence of emotional responsiveness as dissociative symptoms.

Presently the state-of-the art techniques provide no (or only nominally useful) predictors for the initial selection of the antidepressant treatment once refractory depression had been identified. The choice of drug is typically chosen on the basis of safety and convenience, not differential efficacy. The search for the clinical and biological correlates of long-term or acute outcome present a major nosological conundrum: Who will respond to treatment? Which treatment?

In this manner, treatment resistant depression challenges the prognostic utility of our current phenomenological-based diagnostic system. Treatment resistant depression is neither a clinically identifiable entity, nor a biologically identifiable entity. Other treatment interventions that have been recommended for treatment resistant depression include: insulin therapy; Yohimbine augmentation of fluvoxamine; rapid-rate transcranial magnetic stimulation; vagus nerve stimulation; and augmentation of paroxitine with naltrexone (an oral opiate antagonist). In addition, opiates, both pure antagonists and mixed agonist-antagonists have also been reported to be effective treatments of refractory depression.

What is needed, then, is a method for treating refractory depression characterized by dissociation by administering an opiate antagonist and an antidepressant. Such a method is currently unavailable in the art.

### BRIEF SUMMARY OF THE INVENTION

A method of treating a patient with refractory depression characterized by dissociation is disclosed. The method comprises the steps of providing a patient in need thereof (diagnosed with refractory depression characterized by dissociation) with one or more opiate antagonists as well as at least one antidepressant. It is the object of the present invention to provide a novel method of treating refractory depression characterized by dissociation.

It is the object of the present invention to provide a method for treating refractory depression characterized by dissociation by administering to a patient in need thereof an effective dissociation-reversing amount of an opiate antagonist or a pharmaceutically acceptable salt thereof; and an effective depression-reversing amount of an antidepressant or a pharmaceutically acceptable salt thereof.

It is the object of the present invention to provide a method for treating refractory depression characterized by dissociation using an opiate antagonist having a pentacyclic nucleus.

It is the object of the present invention to provide a method for treating refractory depression characterized by dissociation by administering an opiate antagonist that is selected from the group consisting of nalmefene, naloxone, naltrexone, nalbuphine, thebaine, and combinations thereof.

It is the object of the present invention to provide a method for treating refractory depression characterized by dissociation by oral administration of an opiate antagonist.

It is the object of the present invention to provide a method for treating refractory depression characterized by dissociation by administering an effective dissociation-reversing amount of nalmefene. An effective dissociation-reversing amount of nalmefene ranges between about 50 mg. to about 300 mg. b.i.d. Preferably the effective amount of nalmefene comprises an initial dosage of nalmefene in the amount of about 50 mg. b.i.d. for about three days, followed by a dosage of about 100 mg. b.i.d. for about four days, followed by a dosage of about 150 mg. b.i.d. for about one week, followed by a dosage of about 200 mg. b.i.d. per week thereafter until the patient has achieved a dissociation-free state.

It is the object of the present invention to provide a method for treating refractory depression characterized by dissociation wherein the opiate antagonist or pharmaceutically acceptable salt thereof is administered in combination with a pharmaceutically acceptable carrier.

It is the object of the present invention to provide a method for treating refractory depression characterized by dissociation wherein the carrier is selected from the group consisting of water, milk, fruit juice and sweetened beverage.

It is the object of the present invention to provide a method for treating refractory depression characterized by dissociation by administering to a patient in need thereof an antidepressant or pharmaceutically acceptable salt thereof in combination with a pharmaceutically acceptable carrier.

It is the object of the present invention to provide a method for treating refractory depression characterized by dissociation by administering an antidepressant. The antidepressant may be selected from the group consisting of, but not limited to, monoamine oxidase (MAO) inhibitor, tricyclic antidepressant, serotonin reuptake inhibitor, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and combinations thereof.

It is the object of the present invention to provide a method for treating refractory depression characterized by dissociation wherein the antidepressant is selected from the group consisting of, but not limited to, amitriptyline, lofepramine, bupropion, citalopram, fluoxetine, fluvoxamine, imipramine, paroxetine, sertraline, mirtazapine, venlafaxine, nefazodone, nortriptyline, reboxetine, SAM-E and combinations thereof.

It is the object of the present invention to provide a method for treating refractory depression characterized by dissociation wherein the effective depression-reversing amount comprises an initial dosage of Bupropion SR, preferably in the amount of about 200 mg. to about 400 mg. in divided doses.

It is the object of the present invention to provide a method for treating refractory depression characterized by dissociation wherein the effective depression-reversing amount comprises a dosage of Venlafaxine. Preferably the venlafaxine of choice is EFFEXOR® XR in the amount of about 75 mg. per day to about 375 mg. one time daily.

It is the object of the present invention to provide a method for treating refractory depression characterized by dissociation by administering to a patient in need thereof at least one opiate antagonist, evaluating the patient for a response to the opiate antagonist, reassessing the patient for depression, and administrating at least one antidepressant to the patient.

While the following terms are believed to be well understood by one of skill in the art, the following definitions are set forth to facilitate explanation of the invention.

The term “health care provider” is meant to refer to physicians, family practitioners, psychiatrists, psychologists, psychoanalysts, social workers, nurses or any other professional who provides health care services, particularly health care services that identify patients having refractory depression characterized by dissociation.

The term “refractory depression” and treatment-resistant depression are synonymous meaning depressions that respond insufficiently to treatment with the standard antidepressants, such as tricyclic antidepressants (TCAs), monoamine oxidase inhibitors (MAOIs), and serotonin selective reuptake inhibitors (SSRIs), given long enough and in adequate dosage.

The term “dissociation” refers to a mental disorder the essential features of which are characterized as the disruption in the usual integrative functions of consciousness, memory, identity, or perception of the environment. The disturbance may be sudden or gradual, transient or chronic. Individuals with dissociative disorders generally experience difficulty with intimacy, however they may function well in a business setting.

The term “administering” means prescribing or providing medication in a dosage form and amount.

The term “dissociation-reversing amount” means a quantity sufficient to diminish, cure, or alleviate dissociation in a patient.

The term “opiate antagonist” means a remedy substance containing or derived from opium that tends to block the action of an opiate (whether taken internally, or manufactured internally in the body).

The term “effective depression-reversing amount” means a quantity sufficient to diminish, cure, or alleviate depression in a patient.

The term “pentacyclic nucleus” means a compound containing a pentacyclic ring system.

The term “pharmaceutically effective carrier” means a relatively non-toxic and relatively stable carrier.

The terms “BID”, “b.i.d.”, and “bid” are synonymous and mean two times daily.

The terms “patient in need” refers to a person diagnosed with refractory depression characterized by dissociation.

### DETAILED DESCRIPTION OF THE INVENTION

The present invention relates to a method for treating a mental condition, more specifically, refractory depression characterized by dissociation. It is understood by those of skill in the art that health care providers play a vital role in identifying a person with refractory depression characterized by dissociation. It is also understood by those of skill in the art that typically only a medical doctor, psychiatrist, or nurse practitioner would administer or prescribe a medication, or combinations of medications to treat an illness such as mental illness. It is to be appreciated that those of skill in the art of administering or prescribing medications understand that the dosages and choice of drug can vary from patient to patient, due to many factors including, but not limited to, age, sex, weight, general health, allergies, substance abuse, prior medical histories, and prior mental health history. Accordingly, one of skill in the art has clinical experience and can readily modify the presently disclosed method of treating a patient with respect to dosage and choice of drug.

A patient in need of treatment according to the present invention is a person diagnosed by a health care provider to have treatment-resistant depression characterized by dissociation. Those of skill in the art can appreciate that the diagnostic step of identifying a patient in need of treatment is often a process in itself. It is necessary to observe a person being treated for depression, and evaluate that person for change. Often, since patients react to drugs differently, it is necessary to provide various antidepressant drugs over the course of weeks in order to diagnose a patient with refractory depression.

The present invention specifically relates to a patient that is resistant to treatment of depression, and who dissociates. The essential feature of a dissociative disorder is the disruption in the usual integrative functions of consciousness, memory, identity, or perception of the environment. The disturbance may be sudden or gradual, transient or chronic. People who feel emotionally numb, dead, shut-down, hollow, empty, or who report that they cannot experience feelings have lost the ability to access normal human feelings as part of their conscious waking existence. Intimately associated with this disruption in their conscious experience of human feelings is the disruption in their experience of who they are; their sense of identity. People diagnosed with one of the dissociative disorders typically experience having no feelings. For example, feeling dead has been noted in people with symptoms of depersonalization. People diagnosed with dissociative identity disorder have been reported to have symptoms of numbness. The diagnostic criteria for 308.3 Acute Stress Disorder in the DSM IV (pp. 431-432), lists a subjective sense of numbing, detachment, or absence of emotional responsiveness as dissociative symptoms.

A high percentage of people diagnosed with depression who did not respond (or only partially responded) to conventional antidepressant treatments experienced dissociation with a numbing of their responses. Numbing of general responsiveness is identified in the DSM IV as an important aspect of the diagnosis of posttraumatic stress disorder (PTSD). Numbing, as well as, a method of assessing degrees thereof is further described in Glover, Emotional Numbing: A Possible Endorphin-Mediated Phenomenon Associated with Post-Traumatic Stress Disorders and Other Allied Psychopathologic States, 5 J. Traumatic Stress; No. 4, 643-75 (1992), which is herein incorporated by reference.

Diminished responsiveness to the external world usually begins soon after the onset of a traumatic event. The survivor of a trauma may complain of having markedly diminished interest or participation in previously enjoyed activities, of having markedly reduced emotions (especially associated with intimacy, tenderness, and sexuality). Cognitive disturbances have also been associated with the numbing response, including confusion and disorientation and impaired memory formation, recall, and problem solving. Somatic disturbances such as analgesia and paralysis have been associated with the numbing response.

When numbing presents as a syndrome, it may include symptoms associated with major depression such as diminished energy, interest, and pleasure, cognitive impairments, and preoccupation with death. Moreover, both numb and depressed people may present with a blunted facial expression. Emotional numbing denotes an absence of feelings such as depression, sadness, or guilt. However at different times, it is common for emotionally numb people to shift between experiencing numbness and depression. See S. Ramirez et al., Relationship of Numbing to Alexithymia, Apathy, and Depression, 88 Psychological Reports, 189-200 (2001), herein incorporated by reference.

It is to be appreciated that identifying and diagnosing individuals with refractory depression characterized by dissociation, as well as assessing, and reassessing a patient during treatment according to the present invention will be necessary. Known techniques including the Glover Numbing Scale which provides an indicator or measure of numbness and dissociation, as well as the Beck Depression Inventory, which provides an indicator, or measure for depression.

According to one embodiment of the present invention, a patient presenting symptoms of depression is diagnosed with depression by a clinician according to a known test for measuring depression. In one embodiment, the test utilized is one of the Beck Depression Inventory, although it should be apparent that other tests for measuring depression known in the art may also be effectively used to diagnose the patient with depression.

Although any known test for measuring depression may be used as an indicator or measure of depression, the Beck Depression Inventory (“BDI”) is a preferred test for the present invention. The Beck Depression Inventory is a questionnaire developed by Aaron T. Beck, M.D. in 1978 to measure the presence and severity of depression. This test provides an exemplary method of measuring depression over the course of treatment according to the present invention. This test can also be used for reassessing a patient during treatment. The reassessment may be used to determine choice and dosage of antidepressant.

The second edition of the Beck Depression Inventory is a 21-item questionnaire with each item rated on a four-point Likert scale (0-3) with total scores ranging from 0 to 63. A higher score indicates a more severe level of depression. Generally, a total score below 10 indicates no depression; a score from 10-16 indicates mild depression; 17-29 indicates moderate depression; and 30-63 indicates severe depression. According to the present invention, a score of about 10 or above would indicate diagnosis and treatment according to the present invention, with the patient more preferably presenting a BDI score of about 17 or above. A BDI score of less than about 10 is clinically non-significant.

Eleven items of the BDI specifically identify how a depressed person thinks and feels about himself or herself. These items were found to be unique and distinctive for the experience of depression and unrelated to the experience of dissociation. In paraphrased form they are: feeling disappointment in oneself; critical of oneself; suicidal thoughts; sadness; discouraged about the future; feelings of being punished; guilty; feeling like a failure; feeling a lack of satisfaction; feeling old/unattractive/ugly; and loss of interest in people. See S. Ramirez, supra.

Without wishing to be bound by theory, it is thought that the identification of the co-occurrence of depression and dissociation symptoms at the outset of treatment explains the need to administer both an antidepressant and an opiate antagonist medication, and to monitor behavioral responses to both types of medications with scales that measure both symptoms of depression and dissociation. This embodiment of the present invention provides researchers in the field of depression the rationale and the tools to study a clearly identified subgroup of depressed individuals who are refractory to treatment.

Prior to the instant invention, such a diagnosis of depression would have likely resulted in treatment with antidepressant medication; however, as stated above, immediately proceeding with a known antidepressant treatment frequently does not result in a positive response from the patient. That is, the depression can be refractory, unbeknownst to the clinician diagnosing the depression. Previously, the only known way to determine this refractory character was to prescribe an antidepressant and monitor the patient for improvement.

However, according to an embodiment of the present invention, treatment with antidepressant drugs does not immediately follow the diagnosis of depression. Rather, in a preferred embodiment, a questionnaire according to the Glover Numbing Scale (“GNS”) is completed by the patient diagnosed with depression, and depending on the results, the patient may be immediately diagnosed with refractory depression characterized by dissociation.

The Glover Numbing Scale was constructed to measure symptoms associated with the numb responses, and provides an adequate measure for determining whether a patient is in a dissociated state. Both males and females with major depression evidenced a bimodal distribution of scores on the GNS with a distance of three standard deviations between their respective means. The GNS has identified a unique subgroup within the population of people diagnosed with major depression who manifest the unique phenomenological experience and associated symptoms of having no feelings. The Glover Numbing Scale provides an exemplary measure or indicator for dissociation; hence practitioners are capable of evaluating a patient for response to an opiate antagonist. By evaluating a patient with the Glover Numbing Scale prior to administration of an opiate antagonist, and reassessing the patient after treatment, one of skill in the art can readily determine if the opiate antagonist is reversing or alleviating dissociation.

Additionally, evaluating a patient with the Glover Numbing Scale according to the present invention will result in an accurate identification of the condition of refractory depression characterized by dissociation, at the very outset of treatment, thereby preventing the initiation of a futile antidepressant treatment prior to treating the refractory nature of the depression, i.e., the state of dissociation suffered by the patient. Administering both an antidepressant and an opiate antagonist at the initiation of treatment will save these patients and their families' considerable time, money, and unnecessary suffering.

In one embodiment, the Glover Numbing Scale is a 35-item self-report questionnaire that systematically measures the frequency of experiences associated with “numbness,” an essential symptom of dissociation. Each item is rated on a seven-point scale by the patient, with scores corresponding to the following responses: 1, never; 2, very rarely; 3, rarely; 4, occasionally; 5, frequently, 6, very frequently; and 7, always. The highest possible score according to the Glover Numbing Scale is 245 (35 items by 7 points per item); a higher score indicates a more persistent degree of numbness and associated symptoms of dissociation. A study disclosed that 353 male Vietnam combat veterans diagnosed with Post Traumatic Stress Disorder had a mean Glover Numbing Scale of 165.40, with a standard deviation of 34.60. See S. Ramirez, supra at 194-95. According to the present invention, a Glover Numbing Scale score greater than about 100 should be considered consistent with the presence of dissociation. In one embodiment, a GNS score of about 140 or greater indicates that the patient suffers from dissociation. In another embodiment, a GNS score less than about 60 can be considered clinically non-significant.

In addition to using the Glover Numbing Scale, identifying supplementary terms, which are synonymous to the terms used on the Glover Numbing Scale, may also be used to evaluate and reassess a patient for dissociation. For example, identifying whether a patient is “numb”, “hollow”, or “lacks feeling” may provide additional input when evaluating a patient's degree of dissociation. Useful synonyms to the Glover Numbing Scale include absence of feelings, numb, dead, hollow, empty, or lack of feelings. It is to be appreciated that a practitioner will ask the patient questions such as “Do you feel numb or hollow?”

Once identified, a patient in need of treatment would be a depressed person having the dissociative symptom of the inability to access normal feelings in consciousness (e.g. feeling numb, dead, hollow, empty, shutdown, or having no feelings). Many persons with symptoms of dissociation will acknowledge that any one of the enumerated symptoms above adequately describes their subjective experience. Others, however, may only be able to acknowledge one or two of the symptoms as relevant to their condition. Clinicians are encouraged to question patients which term best describes their inner subjective state, and to include that term in their follow-up assessments.

That is, according to one embodiment of the present invention, a patient evaluated according to the Beck Depression Inventory to have a total BDI of about 10 or more, would be diagnosed as suffering from depression. According to the present invention, the depressed patient would then be evaluated according to the Glover Numbing Scale to assess the refractory nature of the patient's depression. If the patient scored about 100 or above, and positively to any of the eight salient items on the GNS, the patient is then affirmatively diagnosed as suffering from refractory depression characterized by dissociation according to one embodiment of the present invention. According to the present invention, the diagnosis may be made immediately after evaluating the patient according to the Glover Numbing Scale in conjunction with the Beck Depression Inventory in the context of an initial diagnostic assessment. The 35 items of the Glover Numbing Scale (GNS) describe the breadth of problems that can be associated with dissociation. Eight of the 35 items, however, uniquely discriminate dissociative experiences from symptoms of depression at a very high degree of probability. It is recommended that these eight items be scored separately at the time of the initial evaluation, and be used to monitor the patient's response to treatment at regular intervals until the patient is in full remission or has achieved the maximum beneficial response to medication.

In one embodiment, a full remission indicates that a patient answers “never” to each of the eight salient items. At the time of remission the patient's responses to all of the other 17 items on the GNS can be individually assessed by the clinician to ascertain if any residual symptoms are present which may require further attention. In one embodiment, the following exemplary eight salient items can be used: (1) “Others tell me I look upset or angry or sad, and I do not know what they are talking about;” (2) “A wall exists between other people and me;” (3) “When I get angry, I feel destructive;” (4) “My body feels numb;” “(5) I pretend I have feelings when I really don't;” (6) “When I get angry I care who might get hurt;” (7) “I feel empty inside;” and (8) “I feel dead or shutdown.” It is understood that items addressing the same subject matter as the above exemplary salient items can be substituted for some or all of the exemplary salient items. A preferred synonym such as “I feel numb” or “I feel hollow” or “I have no feelings” can be substituted for the last two salient items.

According to an embodiment of the present invention, the condition of refractory depression characterized by dissociation may be identified by administering the Glover Numbing Scale (including reference to the above-mentioned eight salient items) to establish a GNS dissociation score, which can be considered in conjunction with the BDI score and any other clinical assessment. In one embodiment, at least one of the eleven salient items of the Beck Depression Index unique to depression (cited in S. Ramirez, et al., supra, Table 2 at 196) should contribute to the BDI depression score.

According to the present invention, this diagnosis may be made very soon after evaluating the patient according to the Glover Numbing Scale. The diagnosis is preferably made less than one week after the GNS evaluation, more preferably within four days of the GNS evaluation, and most preferably within about one day of the GNS evaluation.

Although not wishing to be bound by any theory, it is believed that the Glover Numbing Scale and the Beck Depression inventory are the preferred tests for measuring treatment-resistant depression and dissociation for these tests both require the patient or subject to elicit a response. It is believed that tests, which require the patient to elicit a response, provide more consistent results and a superior indication of dissociation and depression levels.

In one embodiment, after a diagnosis of refractory depression characterized by dissociation has been made, an opiate antagonist can be administered to the patient in order to alleviate the refractory character of the depression caused by the patient's dissociative state.

The opiate antagonist is administered to reverse the dissociation and the antidepressant is administered for residual classic symptoms of depression, which may remain despite the opiate antagonist. It is believed that it is the opiate antagonist, therefore, that reverses an important factor contributing to the refractory nature of the depression, making the condition more typical and amenable to conventional antidepressants. Although any antidepressant may prove helpful, SSRI's, in some individuals, may act to dampen down feelings too much, leading to the condition of apathy and/or absence of feelings. Each person's history of dissociation and depression is unique and will require careful consideration by the clinician to determine the duration of continued administration of both drugs on a maintenance basis.

Although any opiate antagonist would function according to the present invention, the preferred opiate antagonist is an opiate antagonist having a pentacyclic nucleus, preferably nalmefene. Other useful examples of opiate antagonists include naloxone, naltrexone, nalbuphine, and thebaine.

The opiate antagonist can be administered to the patient by any known drug delivery method (such as transdermal, nasal, or intramuscular), however, oral administration is preferred. The opiate antagonist may be combined with any pharmaceutically acceptable carrier. For example, suitable carriers include water, milk, fruit juice and sweetened beverage.

According to one embodiment of the present invention, nalmefene is the preferred opiate antagonist. The patient in need thereof is orally administered between about 50 mg. to about 300 mg. b.i.d. until dissociation is alleviated or reversed. Preferably, the nalmefene is administered in an initial amount of about 50 mg. b.i.d. for the period of about three days, followed by a dosage of about 100 mg. b.i.d. for about four days, followed again by a dosage of about 150 mg. b.i.d. for a period of about one week. One of skill in the art may increase the dosage by about 10 mg. to about 20 mg. per week thereafter until the person has achieved a dissociation-free state or a sufficiently diminished level of dissociation to make the patient receptive to treatment with an antidepressant.

It is to be understood that according to an embodiment of the present invention, one of skill in the art may regularly assess the patient's dissociation level during this treatment period at any time and alter the dosages to provide the optimal effective dissociation-reversing amount of opiate antagonist to the patient. For example, according to an embodiment of the present invention, a patient diagnosed with depression can be assessed for symptoms of dissociation using, for example, a Glover Numbing Scale questionnaire and subsequently measured for the total score and the salient items. Where the number is deemed sufficiently high to indicate the likelihood of dissociation, the clinician will then prescribe a course of treatment with an opiate antagonist, such as the course described above.

Subsequent to opiate antagonist treatment, the patient may be again administered and scored on the Glover Numbing Scale. According to the present invention, this second administration of the GNS is generally completed one week after beginning the opiate antagonist treatment. This second score is compared to the patient's first score on the scale to determine if there has been a response and if so how much.

One skilled in the art will recognize that the time for administering the GNS relates to the prescribed opiate antagonist treatment. Preferably, the second assessment will be assigned soon after the opiate antagonist treatment begins, thereby allowing the clinician the opportunity to evaluate the effects of the prescribed dosage. For example, the patient may be initially evaluated about four days or one week after the opiate antagonist treatment commences. It should be apparent to one skilled in the art that multiple subsequent assessment scores on the GNS may be collected at regular intervals to monitor the response to treatment over time with increasing dose administrations.

According to the present invention, when the patient's level of dissociation as indicated by the score on the Glover Numbing Scale becomes clinically insignificant, or otherwise improves such that the patient's depression is no longer refractory, the patient is preferably administered an effective depression-reversing amount of an anti-depressant in order to treat the underlying diagnosed depression. Alternatively, where an initial depression-score according to the BDI is sufficiently high to indicate a likelihood of depression, the patient may be administered an anti-depressant simultaneously with the opiate antagonist.

It is to be appreciated that there are many drugs and methods available for treating depression. The present invention combines known methods of treating depression with an opiate antagonist to solve the problem of refractory depression characterized by dissociation. Hence, there are many suitable antidepressants for use in accordance with the present invention. The antidepressants can be administered to the patient by any conventional drug delivery method, however, oral administration is preferred. Suitable antidepressants include monoamine oxidase (MAO) inhibitors, tricyclic antidepressants, serotonin reuptake inhibitors, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and combinations thereof.

Specific examples of suitable antidepressants include amitriptyline, lofepramine, bupropion, citalopram, fluoxetine, fluvoxamine, imipramine, paroxetine, sertraline, mirtazapine, reboxetine, venlafaxine, nefazodone, nortriptyline, SAM-E and combinations thereof.

Table one shows a list of suitable antidepressants, all of which may be administered according to known methods such as those methods shown and described in the Physicians' Desk Reference, 55th Edition (2001), herein incorporated by reference.

TABLE 1
Nardil and Parnate (monoamine oxidase, or MAO, inhibitors)
Anafranil (clomipramine), Asendin (amoxapine), Aventyl and Pamelor
(nortriptyline), Elavil (amitriptyline), Norpramin (desipramine), Sinequan
(doxepin), Surmontil (trimipramine), Tofranil (imipramine), and Vivactil
(protriptyline). (Tricyclic compounds)
Ludiomil, Maprotiline, and Remeron (tetracyclic compounds)
Wellbutrin, Zyban (bupropion)
Desyrel (trazodone)
Prozac, Zoloft, and Paxil (selective serotonin re-uptake inhibitors,
or SSRI's)
Effexor (venlafaxine)
Serzone (nefazodone)

According to another embodiment of the present invention, Bupropion SR is the preferred antidepressant. The patient in need thereof is administered between about 200 mg. to 400 mg. in divided doses.

According to another embodiment of the present invention, Venlafaxine (such as Effexor® SR) is the preferred antidepressant. The patient in need thereof is administered between about 75 mg. per day to about 375 mg. per day, one time daily.

There are at least three major opioid receptor types in the central nervous system (hereinafter CNS) and in the periphery. These receptors, known as mu, delta, and kappa, have distinct pharmacological profiles, anatomical distributions and functions. The delta receptors are abundant in CNS and mediate analgesia, gastrointestinal motility and various hormonal functions. The mu receptors bind morphine-like drugs and mediate the opiate phenomena associated with morphine, including analgesia, opiate dependence, cardiovascular and respiratory functions, and several neuroendocrine effects.

Although not wishing to be bound by any theory of how the present invention works, it is believed that the opiate antagonists of the present invention have an affinity for kappa receptors. The kappa receptors have the widest distribution in CNS and mediate a spectrum of functions including the modulation of drinking, water balance, food intake, gut motility, temperature control and various endocrine functions. They also produce analgesia. Although not wishing to be bound by any theory, it is believed that opiate antagonists of the present invention provide the greatest dissociation reversing effect when bound to kappa receptors; hence a more specific opiate blocker is able to target a more specific receptor type.

The following examples are given for the purpose of illustrating the present invention and are not intended to limit the scope in any way.

### EXAMPLE 1

Mr. A is a 68 year-old divorced, retired, over nourished Caucasian male of average height with a high school education. He was initially evaluated for symptoms of depression and social withdrawal following the death of his best friend during the previous year. Six months after the death he had been unsuccessfully administered Paxil (a serotonin selective reuptake inhibitor [SSRI) up to 60 mg./day. Paxil did provide a mild to moderate calming effect. Bupropion, (sustain released), administered up to 300 mg./day was added to the paxil with little benefit other than some improvement in his level of energy. Both antidepressants were stopped and he was then administered desipramine (a tricyclic antidepressant) up to 150 mg./day for 4 weeks. Mr. A reported some decrease of crying spells, but remained essentially depressed and sad, with diminished interest and pleasure, impaired concentration and memory. At times his sleep and appetite were increased and other times they were decreased. Desipramine was discontinued and Mr. A was started on a course of electric shock treatment (ECT) on an outpatient basis. He received a total number of 8 treatments, administered 2 times per week. Mr. A experienced a good response with improved mood, appetite, sleep, and pleasure and interest and motivation for a period of 6 weeks before symptoms of depression, once again, gradually returned.

In the past, Mr. A had been diagnosed with unipolar depression, recurrent type, of a nonpsychotic nature. In the past he had always enjoyed good responses to antidepressants (such as paxil and desipramine).

The patient's medical history included adult onset type II diabetes mellitus which was controlled with oral hypoglycemic agents and diet. He had a history of alcohol abuse, but had been abstinent for the last 3 years. He complained of intermittent joint pain associated with osteoarthritis, but was not taking any anti-inflammatory medication.

Six weeks after completing his last ECT treatment, Mr. A appeared depressed, and at times, tearful. He experienced some difficulty putting feelings into words. When asked directly, he acknowledged feeling periodically emotionally dead and empty. Physical examination including fasting blood sugar and thyroid tests (TSH, T3, T4) were at normal values. His score on the Beck Depression Inventory (BDI) was significantly high (23). His total score on the Glover Numbing Scale (GNS) was 142. Mr. A's responses to 6 of 8 salient items on the GNS clearly indicated that he experienced numbing on a regular basis. These items included: “I feel dead or shut down,” “My body feels numb,” “When I get angry I feel destructive,” “A wall exists between other people and me,” “Others tell me I look upset or angry or sad, and I don't know what they are talking about,” “I pretend that I have feelings when I really don't.” The patient was diagnosed with refractory depression characterized by dissociation.

The patient was begun on nalmefene 50 milligrams b.i.d. for three days, then increased to 100 mg. b.i.d. the next 4 days, and then increased to 150 mg. b.i.d. Side effects of drowsiness and tiredness were mild and transient in nature. He was reassessed with the BDI and the GNS. One week later Mr. A was able to identify his mood as sad and lonely. He no longer experienced any lack of feelings. His BDI was now scored at 20 and his GNS score fell to 70. Salient items were reported to not have been experienced during the past week.

Mr. A. was started on SAM-e 400 mg. in divided doses before breakfast and lunch, which was increased to 800 mg. five days later. One week afterward he reported a marked improvement in mood and energy level. His GNS score further reduced to 40 and the BDI to 6. Both scores are clinically non-significant. Mr. A. appeared bright, alert, responsive, and interested in his usual activities which provided him pleasure and satisfaction. He felt reconnected to the outside world and others.

### EXAMPLE 2

Mrs. B is a 38 year-old Caucasian who is a married, college educated female employed as an office manager. Mrs. B is of average height and weight, without any medical problems other than irregular menses during the past two years. The past two years Mrs. B wrestled with a number of stressors including her husband's infidelity, sexual harassment in the workplace, and caring for a terminally ill mother with cancer.

Mrs. B. described a 14-month history of depression, withdrawal, sadness, loss of interest and motivation. She frequently alluded to feeling that something inside of her was missing. She had lost the ability to cry. Sleep and appetite were disturbed. She frequently experienced a lack of care and concern for herself and others. Sometimes she felt empty and sad, and other times empty and having no feelings. Friends remarked that, on occasion, she was uncharacteristically irritable, insensitive to others, and flippant.

Mrs. B was initially treated with Prozac, which had the beneficial effect of reducing irritability. Otherwise, she remained unchanged. Lithium carbonate 300 mg. b.i.d. was added to the Prozac for one week, and afterward Prozac was increased over the next 4 weeks to 60 mg./day. Serum lithium levels were reported as 0.6 and 0.7. Blood tests showed a normal thyroid profile (tsh, t3, t4, cell blood count, and chemistry). A lack of response convinced both doctor and patient to discontinue lithium and Prozac over the next 4 weeks. In succession, she was tried on amitriptyline up to 300 mg./day with monitoring of medication blood levels, and venlafaxine up to 375 mg./day. Both medications improved her level of energy and had a calming effect with some reduction of sadness and emptiness.

Mrs. B was administered the GNS and BDI and scored respectively 148 and 24. Salient items were reported as frequent events during the past week. Her facial expression and mood were observed to be labile. She was tearful, and at times sharp and irritable. Other times she showed no emotion at all when sharing very troubling information. Mrs. B was diagnosed with treatment resistant depression characterized by dissociation.

It was decided to maintain the patient on amitriptyline while introducing nalmefene 50 mg. b.i.d. Four days later the dose of nalmefene was doubled. Mrs. B was monitored for side effects and changes of mood, using the eight salient items of the GNS and the eleven salient items of the BDI as guides. Mrs. B complained of side effects associated with the drug amitriptyline. During each visit the dose was reduced 50 mg. to a final dose of 200 mg./day. Mrs. B volunteered that she began to feel the antidepressant effect for the first time.

One week after the dose of nalmefene 200 mg./ b.i.d. had been reached, Mrs. B was reassessed. She reported that she felt her usual self once again—vibrant and alive to the world. She regained feelings of care and concern for others. GNS and BDI scores were 38 and 5, respectively. Salient items of the GNS were reported not to have been experienced. At this particular junction, Mrs. B was ready and able to deal with the stresses in the marriage and in the work place. She agreed to continue taking both medications and to begin counseling with a psychotherapist.

It is to be appreciated that the foregoing is illustrative and not limiting of the invention, and that various changes and modifications to the preferred embodiments described above will be apparent to those skilled in the art. Such changes and modifications can be made without departing from the spirit and scope of the present invention, and it is therefore intended that such changes and modifications be covered by the following claims.

What is claimed as new and desired to be protected by Letters Patent of the United States is:

1. A method for identifying refractory depression characterized by dissociation, comprising:
identifying a patient with depression; and
evaluating said patient for dissociation according to the Glover Numbing Scale by administering the Glover Numbing Scale and generating a dissociation score, wherein the dissociation score indicates said patient has refractory depression characterized by dissociation.

2. The method of claim 1, wherein said step of diagnosing a patient with depression comprises evaluating said patient according to the Beck Depression Inventory.

3. The method of claim 1, wherein said dissociation score indicates said patient has dissociation.

4. The method of claim 1, wherein said identification of refractory depression characterized by dissociation is made within about one week after evaluating said patient according to the Glover Numbing Scale and the Beck Depression Inventory.

5. The method of claim 4, wherein said identification of refractory depression characterized by dissociation is made within about four days after evaluating said patient according to the Glover Numbing Scale and the Beck Depression Inventory.

6. The method of claim 1 further comprising the step of administering to said patient an effective dissociation-reversing amount of an opiate antagonist or a pharmaceutically acceptable salt thereof.

7. The method of claim 6, wherein said opiate antagonist has a pentacyclic nucleus.

8. The method of claim 7, wherein the opiate antagonist is selected from the group consisting of: nalmefene, naloxone, naltrexone, nalpuphine, thebaine, and combinations thereof.

9. The method of claim 6, wherein the opiate antagonist is selected from the group consisting of at least one kappa opiate antagonist.

10. The method of claim 6, wherein the route of administration is selected from the group consisting of transdermal, nasal, intramuscular, oral, intravenous, parenteral, and inhalation.

11. The method of claim 6, wherein said step of administering comprises administering two doses per day of about 50 mg. of nalmefene for about three days, followed by two doses per day of about 100 mg. nalmefene for about four days, followed by two doses per day of about 150 mg. nalmefene for about one week, followed by two doses per day of about 200 mg. nalmefene thereafter until said patient has achieved a substantially dissociation-free state.

12. The method of claim 6, wherein the opiate antagonist or pharmaceutically acceptable salt thereof is administered in combination with a pharmaceutically acceptable carrier.

13. The method of claim 12, wherein the carrier is selected from the group consisting of water, milk, fruit juice and sweetened beverage.

14. The method of claim 6 further comprising the step of evaluating said patient for a response to said opiate antagonist according to the Glover Numbing Scale.

15. The method of claim 14, wherein the step of evaluating said patient for a response to said opiate antagonist according to the Glover Numbing Scale further comprises the step of evaluating said patient for responses selected from the group of synonyms consisting of numbness, hollow, lack of feeling, and combinations thereof.

16. The method of claim 6 further comprising the step of administering to said patient an effective depression-reversing amount of an antidepressant or a pharmaceutically acceptable salt thereof.

17. The method of claim 16, wherein said step of administering an antidepressant follows a determination that said patient is in a substantially dissociation-free state according to the Glover Numbing Scale.

18. The method of claim 17, wherein said step of administering an antidepressant occurs simultaneously with an administration of a dissociation-reversing amount of an opiate antagonist.

19. The method of claim 17, wherein the antidepressant or pharmaceutically acceptable salt thereof is administered in combination with a pharmaceutically acceptable carrier.

20. The method of claim 17, wherein the antidepressant is selected from the group consisting of monoamine oxidase (MAO) inhibitor, tricyclic antidepressant, serotonin reuptake inhibitor, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and combinations thereof.

21. The method of claim 20, wherein the antidepressant is selected from the group consisting of amitriptyline, lofepramine, bupropion, citalopram, fluoxetine, fluvoxamine, imipramine, paroxetine, sertraline, venlafaxine, nefazodone, nortriptyline, mirtazapine, reboxetine, SAM-E and combinations thereof.

22. The method of claim 21, wherein the effective depression reversing amount comprises about 300 mg. to about 400 mg. per day of Bupropion SR in divided doses.

23. The method of claim 21, wherein the effective depression reversing amount comprises at least one dose of about 75 mg. to about 375 mg. per day of Venlafaxine.

24. A method for treating refractory depression characterized by dissociation, comprising:
identifying a patient with depression;
evaluating said patient for dissociation according to the Glover Numbing Scale;
determining that said patient suffers from dissociation according to the Glover Numbing Scale; and
administering two doses per day of about 50 mg. nalmefene for about three days, followed by two doses per day of about 100 mg. nalmefene for about four days, followed by two doses per day of about 150 mg. nalmefene for about one week.

25. The method of claim 24, further comprising the step of evaluating said patient according to the Glover Numbing Scale to determine if said patient suffers from dissociation.

26. The method of claim 25, wherein said patient is determined to be in a substantially dissociation-free state after said step of evaluating said patient for a response to said nalmefene according to the Glover Numbing Scale.

27. The method of claim 26, wherein said patient is determined to continue to suffer from dissociation after said step of evaluating said patient for a response to said nalmefene according to the Glover Numbing Scale.

28. The method of claim 25 further comprising the step of administering nalmefene in a dosage of about 200 mg. b.i.d. after said step of evaluating said patient for a response to said opiate antagonist according to the Glover Numbing Scale.

29. The method of claim 28 further comprising the step of periodically evaluating said patient for a response to said 200 mg. b.i.d. dosages of said nalmefene by administering the Glover Numbing Scale until said patient is determined to be in a substantially dissociation-free state after said step of evaluating said patient for a response to said nalmefene according to the Glover Numbing Scale.

30. The method of claim 25 further comprising the step of administering to said patient an effective depression-reversing amount of an antidepressant or a pharmaceutically acceptable salt thereof.

31. The method of claim 30, wherein said step of administering an antidepressant follows a determination that said patient is in a substantially dissociation-free state according to the Glover Numbing Scale.

32. The method of claim 30, wherein said step of administering an antidepressant occurs simultaneously with an administration of a dissociation-reversing amount of an opiate antagonist.

33. The method of claim 30, wherein the antidepressant or pharmaceutically acceptable salt thereof is administered in combination with a pharmaceutically acceptable carrier.

34. The method of claim 30, wherein the antidepressant is selected from the group consisting of monoamine oxidase (MAO) inhibitor, tricyclic antidepressant, serotonin reuptake inhibitor, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and combinations thereof.

35. The method of claim 34, wherein the antidepressant is selected from the group consisting of amitriptyline, lofepramine, bupropion, citalopram, fluoxetine, fluvoxamine, imipramine, paroxetine, sertraline, venlafaxine, nefazodone, nortriptyline, mirtazapine, reboxetine, SAM-E and combinations thereof.

36. The method of claim 35, wherein the effective depression reversing amount comprises about 200 mg. to about 400 mg. per day of Bupropion SR in divided doses.

37. The method of claim 35, wherein the effective depression reversing amount comprises at least one dose of about 75 mg. to about 375 mg. per day of Venlafaxine.

38. A method for determining if the condition of refractory depression characterized by dissociation is reduced in a patient, comprising:
diagnosing a patient with depression;
diagnosing said patient with dissociation by administering and measuring a first dissociation scale score according to the Glover Numbing Scale;
administering to said patient an effective dissociation-reversing amount of an opiate antagonist or a pharmaceutically acceptable salt thereof;
administering and measuring a second dissociation scale score according to the Glover Numbing Scale less than one month after commencing said step of administering said opiate antagonist; and
confirming the abatement of the dissociative condition by comparing said first dissociation scale score with said second dissociation-scale score wherein said dissociative condition is reduced if said second dissociation scale score is lower that the first dissociation score.

39. The method of claim 38, wherein said second dissociation scale score according to the Glover Numbing Scale is measured less than about two weeks after commencing said step of administering said opiate antagonist.

40. The method of claim 39, wherein said second dissociation scale score according to the Glover Numbing Scale is measured less than about one week after commencing said step of administering said opiate antagonist.

41. The method of claim 40, wherein said second dissociation scale score according to the Glover Numbing Scale is measured about four days after commencing said step of administering said opiate antagonist.

42. The method of claim 38, wherein said step of identifying a patient with depression comprises evaluating said patient according to the Beck Depression Inventory.

43. The method of claim 38, wherein said opiate antagonist has a pentacyclic nucleus.

44. The method of claim 38, wherein the opiate antagonist is selected from the group consisting of: nalmefene, naloxone, naltrexone, nalpuphine, thebaine, and combinations thereof.

45. The method of claim 38, wherein the opiate antagonist is selected from the group consisting of at least one kappa opiate antagonist.

46. The method of claim 38, wherein the route of administration is selected from the group consisting of transdermal, nasal, intramuscular, oral, intravenous, parenteral, and inhalation.

47. The method of claim 38, wherein said step of administering comprises administering two doses per day of about 50 mg. of nalmefene for about three days, followed by two doses per day of about 100 mg. nalmefene for about four days, followed by two doses per day of about 150 mg. nalmefene for about one week, followed by two doses per day of about 200 mg. nalmefene thereafter until said patient has achieved a substantially dissociation-free state.

48. The method of claim 38, wherein the opiate antagonist or pharmaceutically acceptable salt thereof is administered in combination with a pharmaceutically acceptable carrier.

49. The method of claim 48, wherein the carrier is selected from the group consisting of water, milk, fruit juice and sweetened beverage.

50. The method of claim 38 further comprising the step of administering to said patient an effective depression-reversing amount of an antidepressant or a pharmaceutically acceptable salt thereof.

51. The method of claim 50, wherein said step of administering an antidepressant follows a determination that said patient is in a substantially dissociation-free state according to the Glover Numbing Scale.

52. The method of claim 50, wherein said step of administering an antidepressant occurs simultaneously with an administration of a dissociation-reversing amount of an opiate antagonist.

53. The method of claim 50, wherein the antidepressant or pharmaceutically acceptable salt thereof is administered in combination with a pharmaceutically acceptable carrier.

54. The method of claim 50, wherein the antidepressant is selected from the group consisting of monoamine oxidase (MAO) inhibitor, tricyclic antidepressant, serotonin reuptake inhibitor, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and combinations thereof.

55. The method of claim 54, wherein the antidepressant is selected from the group consisting of amitriptyline, lofepramine, bupropion, citalopram, fluoxetine, fluvoxamine, imipramine, paroxetine, sertraline, venlafaxine, nefazodone, nortriptyline, mirtazapine, reboxetine, SAM-E and combinations thereof.

56. The method of claim 55, wherein the effective depression reversing amount comprises about 300 mg. to about 400 mg. per day of Bupropion SR in divided doses.

57. The method of claim 55, wherein the effective depression reversing amount comprises at least one dose of about 75 mg. to about 375 mg. per day of Venlafaxine.
